(19) United States
(12) Patent Application Publication
(10) Pub. No.: US 2011/0257617 A1
(43) Pub. Date: Oct. 20, 2011

(54) Therapeutic Diffusion Hydrocolloid Wound Dressings

(76) Inventor: Amie B. Franklin, Woodbury, MN (US)

(21) Appl. No.: 13/077,381

(22) Filed: Mar. 31, 2011

Related U.S. Application Data
(60) Provisional application No. 61/320,415, filed on Apr. 2, 2010.

(51) Int. Cl. A61F 13/02; B65B 31/00

(52) U.S. Cl. 604/368; 53/434

(57) ABSTRACT

A diffusion dressing is provided comprising a paper liner, a hydrocolloid layer, a first adhesive layer, a first barrier layer with a center cutout, an oxygen release layer, an absorbent layer, a second barrier layer, and a second adhesive layer with a center cutout. In a preferred embodiment the diffusion dressing is used for diffusing oxygen into a wound.

### CROSS-REFERENCE TO RELATED DOCUMENTS

The present invention claims priority to a provisional patent application Ser. No. 61/320,415 filed on Apr. 2, 2010 and incorporates all disclosure from that application at least by reference.

### BACKGROUND OF THE INVENTION

1. Field of the Invention

The present invention is in the field of medical devices and pertains particularly to methods and apparatus for constructing a hydrocolloid diffusion dressing for wound treatment.

2. Discussion of the State of the Art

In the art of wound care, dressings are applied over various types of wounds to aid in healing. More recently, diffusion dressings have been developed that are able to diffuse therapeutic agents into a wound being treated. The inventor is aware of a diffusion dressing that includes a reservoir that may be pre-charged with a therapeutic agent such as oxygen. In particular, oxygen diffusion dressings may be used on a wide variety of wounds that benefit from better exposure to the oxygen.

A problem with current devices is that they have a somewhat limited diffusion rate and healing time. Moreover, such devices lack certain features that could enable optimum wound oxygenation while absorbing wound exudate and keep the wound moist.

Therefore, what is clearly needed is a hydrocolloid diffusion dressing for treating wounds and at-risk tissue.

### SUMMARY OF THE INVENTION

A problem stated above is that extended diffusion capability is desirable for a therapeutic gas diffusion dressing, but many of the conventional means for constructing a therapeutic gas diffusion dressing are not adequate for extended diffusion periods, such as five or more days without the need of changing dressings. The inventors therefore considered functional components of a diffusion dressing, looking for elements and materials that exhibit superior diffusion qualities that could potentially be harnessed to provide a diffusion dressing that enables extended periods diffusion of therapeutic gas but in a manner that would not create more work or complexity of application.

Every therapeutic gas diffusion dressing has a diffusion reservoir pre-charged or pre-chargeable with a therapeutic gas such as oxygen, one by-product of which is a shortened time for the treated wound to heal. Most such diffusion dressings employ multiple layers of specific materials and certain synergistic features brought about by specific combinations of those layers to conduct the therapeutic gases from the diffusion reservoir of the dressing to a wound or area of tissue that requires treatment.

The present inventor realized in an inventive moment that if, at the point of application, therapeutic gasses in the dressing could be caused to diffuse into the wound in a better regulated manner, significant reduction in healing time might result. The inventor therefore constructed a unique hydrocolloid therapeutic diffusion dressing for healing wounds and other tissue conditions that allowed gases to diffuse more steadily and for longer periods, but constrained such therapeutic gases to the marked area of the tissue with little or no leakage from the edges of the dressing. A significant shortening of time to heal results, with no impediment to ease of application created.

Accordingly, in one embodiment of the present invention, a diffusion dressing is provided comprising a paper liner, a hydrocolloid layer, a first adhesive layer, a first barrier layer with a center cutout, an oxygen release layer, an absorbent layer, a second barrier layer, and a second adhesive layer with a center cutout. In a preferred embodiment the diffusion dressing is used for diffusing oxygen into a wound. In this embodiment, the paper liner is removed to expose the hydrocolloid layer before application. In other embodiments, the diffusion dressing is used as a cover dressing for gauze, alginate, or hydrogel dressings.

In one embodiment of the invention, one or more of the layers is perforated to regulate diffusion rate. In one embodiment, one or more of the layers is pre-treated with silver oxide. In one embodiment, the paper liner and the hydrocolloid layer are perforated, the perforations symmetrically aligned between the layers. In a variation of this embodiment, the perforations in the hydrocolloid layer are larger in diameter than the perorations in the paper liner. In a preferred embodiment, the diffusion dressing is vacuum evacuated and oxygen flushed within an oxygen barrier pouch before application. In all embodiments, the first and second barrier layers form an oxygen reservoir.

According to one aspect of the present invention, a method is provided for charging an oxygen hydrocolloid diffusion dressing comprising steps (a) placing the dressing inside a oxygen barrier pouch, (b) vacuum evacuating the oxygen barrier pouch with the dressing inside, (c) flushing the pouch with oxygen, and (d) translating oxygen from the pouch into the reservoir of the dressing. In a preferred aspect of the method, the oxygen hydrocolloid dressing comprises a paper liner, a hydrocolloid layer, an adhesive layer, a first barrier layer with a center cutout, an oxygen release layer, an absorbent layer, a second barrier layer without cutout, and a non-permeable layer with a center cutout.

In this aspect, in step (b), a vacuum-sealing machine is used to evacuate and flush the pouch with oxygen. In a variation of this aspect, one or more of the layers is perforated to regulate diffusion rate. In another variation of the aspect, one or more of the layers is pre-treated with silver oxide. In one aspect, the paper liner and the hydrocolloid layer are perforated, the perforations symmetrically aligned between the layers. In this aspect, the perforations in the hydrocolloid layer are larger in diameter than the perorations in the paper liner. In a preferred aspect, in step (d), gas from the pouch enters the reservoir of the dressing through one or more gas passages.

### BRIEF DESCRIPTION OF THE DRAWING FIGURES

FIG. 1 is a top view of a hydrocolloid diffusion dressing according to an embodiment of the present invention.

FIG. 2 is a bottom view of the hydrocolloid dressing of FIG. 1.

FIG. 3 is a plan view of a coated paper liner protecting the hydrocolloid dressing of FIG. 1.

FIG. 4 is a plan view of a hydrocolloid layer of the diffusion dressing of FIG. 1.

FIG. 5 is a plan view of an adhesive layer of the hydrocolloid diffusion dressing of FIG. 1.

FIG. 6 is a plan view of a first barrier layer of the hydrocolloid dressing of FIG. 1.

FIG. 7 is a plan view of an oxygen release layer of the hydrocolloid dressing of FIG. 1.

FIG. 8 is a plan view of an absorbent patch of the hydrocolloid dressing of FIG. 1.

FIG. 9 is a plan view of a second barrier layer of the hydrocolloid dressing of FIG. 1.

FIG. 10 is a plan view of a second adhesive layer of the hydrocolloid dressing of FIG. 1.

### DETAILED DESCRIPTION

The inventors provide a unique hydrocolloid diffusion dressing that has an architecture that enables prolonged diffusion of therapeutic gasses such as oxygen into a treated wound, shortening the time to heal. The present invention will be described in enabling detail using the following examples, which may describe more than one relevant embodiment falling within the scope of the present invention.

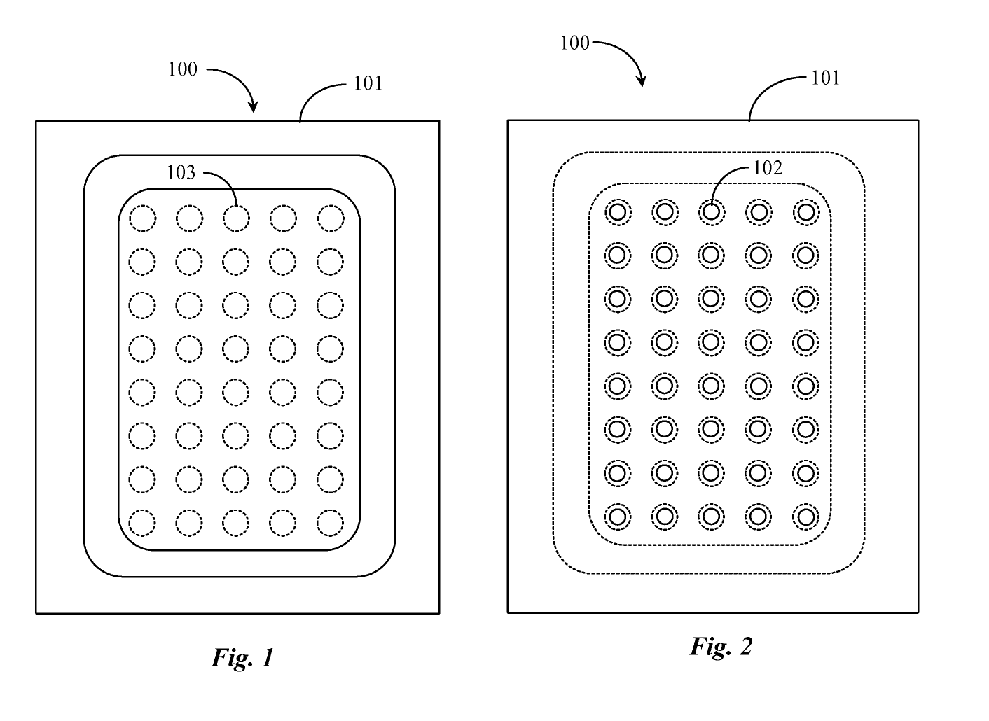

FIG. 1 is a top view of a hydrocolloid diffusion dressing 100 according to an embodiment of the present invention. Dressing 100 is a multi-layer hydrocolloid oxygen diffusion dressing designed to deliver elevated levels of oxygen to a wound site to accelerate healing. Dressing 100 includes an oxygen reservoir (not illustrated) created by sandwiching a layer of open material functioning as an oxygen release layer, between a semi-permeable or non-permeable hydrocolloid diffusion layer and a non-permeable barrier layer. Dressing 100 may be fabricated from a variety of permeable and non-permeable materials or films. Integration thereof may include heat-sealing about the periphery of the dressing to hold the layers together and to form the diffusion reservoir.

In this example, diffusion dressing 100 sits on a paper liner 101 that is coated with a substance that allows quick release of the liner for application of the dressing to a wound or area of tissue undergoing treatment. The permeable film layer may be placed over the wound site and held in place, at its perimeter, with an adhesive border. The adhesive border may be strong enough to keep the film in place for a period of up to five days without allowing air channels to form whereby, they may allow the therapeutic oxygen to escape from the edges of the dressing. Also in this example, the hydrocolloid layer is perforated with diffusion openings 102 arrayed in a symmetrical pattern to aid in even gas distribution during the diffusion process.

FIG. 2 is a bottom view of hydrocolloid dressing 100 of FIG. 1. Hydrocolloid dressing 100 may be used to cover and protect wounds and catheter sites, or may be used as a secondary dressing for covering other wound products, such as gauze, alginates, hydrogels, debridement facilitators or a protective cover. The dressing may be used to treat any area of tissue considered at risk. Moreover, dressing 100 may be indicated for clean closed surgical incisions; skin graft donor sites; Stage I or II pressure ulcers; pressure sores. Dressing 100 may be used to treat superficial wounds such as abrasions; skin tears; blisters; lacerations; first and second-degree burns; chafed skin; or skin continuously exposed to moisture.

Paper liner 101 includes perforations 103 arrayed in a symmetrical pattern that is substantially aligned with the larger perforations 102 in the hydrocolloid layer perforation array of dressing 100. Perforations in the paper liner may aid the per-charging process used to saturate the dressing with therapeutic gas such as oxygen for example. The top non-permeable layer covering the dressing may be of a solid color, transparent or any level of opacity. Similarly, various layers that make up dressing 100 include features that will be described in more detail layer in this specification.

Dressing 100 may be pre-charged in a high oxygen barrier pouch. Pre-charging diffusion dressing 100 involves vacuum evacuation and oxygen flushing using a bench-top vacuum/gas sealer. The grade for oxygen used is, in one embodiment, 99.0% USP grade oxygen. The pouch and dressing will be immersed for a sufficient time to equilibrate at a target oxygen concentration exceeding 90% for active diffusion. To allow proper equilibration the liner of the barrier pouch includes features that allow a gas path from the pouch to the oxygen reservoir in the dressing.

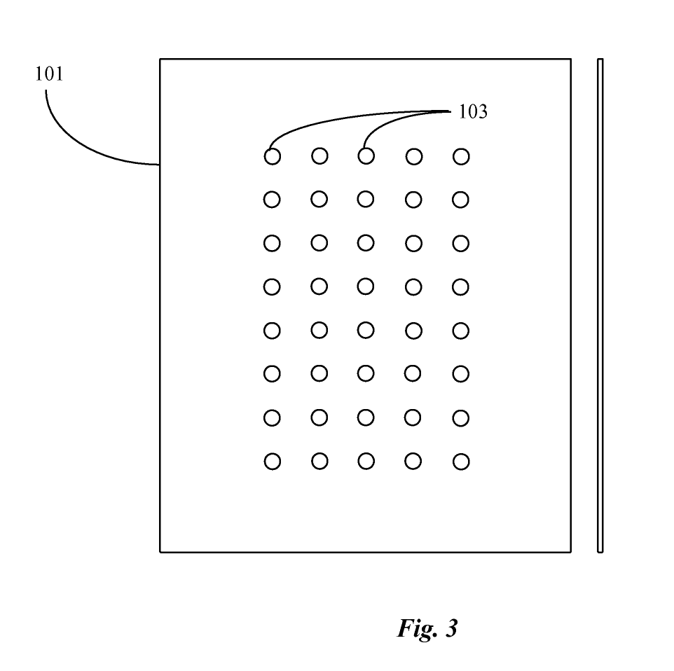

FIG. 3 is a plan view of a coated paper liner 101 protecting the hydrocolloid dressing of FIG. 1. Paper liner 101 includes perforations 103 arrayed in a symmetrical pattern as described further above. These perforations number 40 in this example, however, there may be more or fewer perforations or openings than are illustrated in this example without departing from the spirit and scope of the present invention.

Paper liner 101 has a minimum thickness and is extremely pliable. Paper liner 101 is coated with a non-sticky material to create a peel-back seal for simple activation of the device. In this example, perforations 103 are about 0.25 inches in diameter. However, the diameter of diffusion perforations may be greater or less than 0.25 inches without departing from the spirit and scope of the present invention. The exact size depends on the exact diffusion regulatory requirements attributed to the dressing. Paper liner 101 is illustrated in side view as well in this example to illustrate thickness of the material. In a preferred embodiment, paper liner 101 is about 2 to 5 mils thick, but may be thicker or thinner without departing from the spirit and scope of the present invention. In practice, paper liner 101 may be removed before applying dressing 100 over a wound or tissue area to be treated.

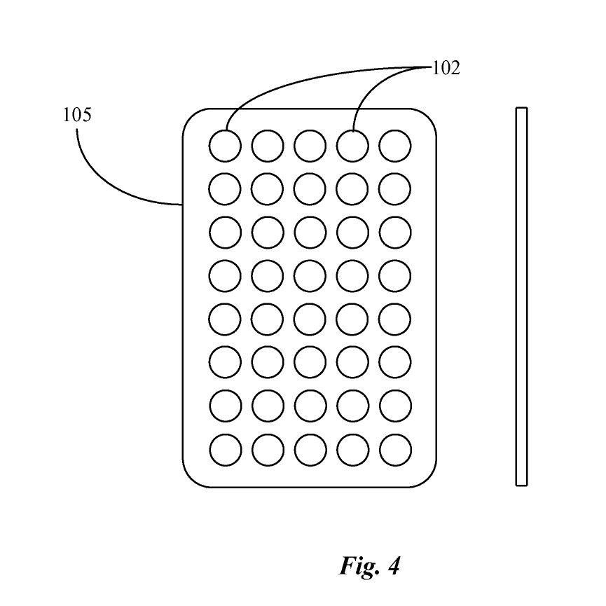

FIG. 4 is a plan view of a hydrocolloid layer 105 of the hydrocolloid diffusion dressing of FIG. 1. Hydrocolloid layer 105 may comprise any suitable substance such as a gum or agar that forms a colloid when mixed with water. Hydrocolloid layer 105 may be impermeable to oxygen such that the diffused oxygen enters the wound through the perorations in the dressing. Hydrocolloid layer 105 may be semi-permeable in one embodiment.

Hydrocolloid layer 105 is also illustrated in side view in this example to illustrate a substantial thickness. In this example, hydrocolloid layer 105 is formed and cut and includes perforations 102. Perforations 102 are about 0.5 inches in diameter. However, in other embodiments, the perforations may be larger in diameter or smaller diameter depending on the exact requirements of the dressing. In this example, hydrocolloid layer 104 has corner radiuses of about 0.25 inches. Corner radiuses may be larger or smaller than 0.25 inches without departing from the spirit and scope of the present invention.

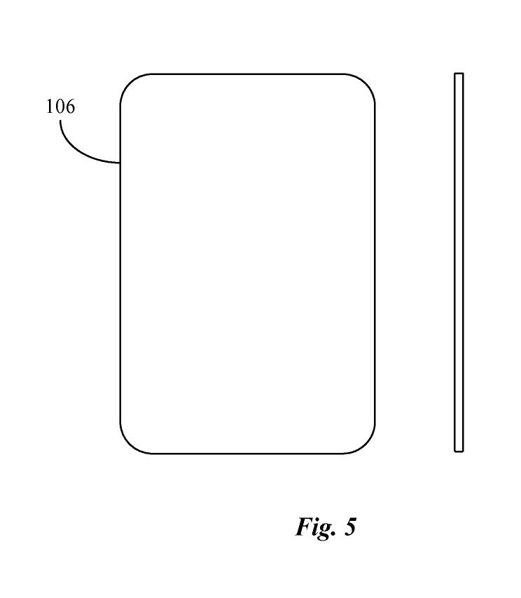

FIG. 5 is a plan view of an adhesive layer 106 of the hydrocolloid diffusion dressing of FIG. 1. Adhesive layer 106 is substantially the same size and thickness as hydrocolloid layer 105 described above. Adhesive layer 106 maintains an adhesive side facing the hydrocolloid layer. Adhesive layer 106 may be semi-permeable or permeable. In one embodiment the adhesive layer is a coated polyurethane film. In this example, the corners of adhesive layer 106 are rounded to 0.25 inches or thereabout. Adhesive layer 106 may be a composite collagen-based material with substantial thickness. In a preferred embodiment, adhesive layer is permeable to therapeutic gasses.

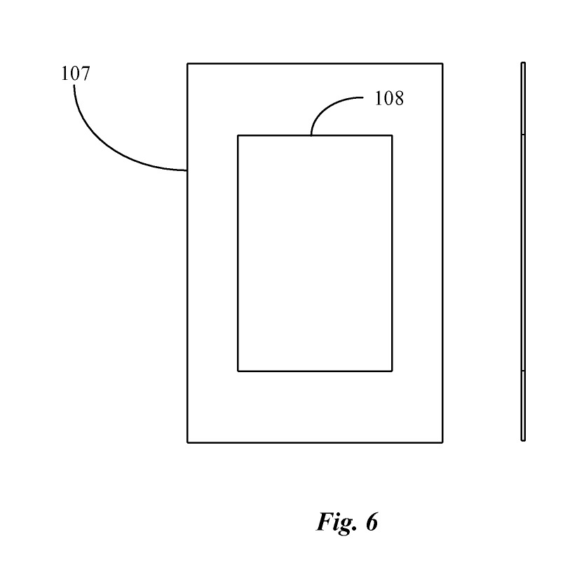

FIG. 6 is a plan view of a first barrier layer 107 of the hydrocolloid dressing of FIG. 1. Barrier layer 107 is a non-permeable poly-based film adapted to provide edge protection in the form of a seal that prevents therapeutic gasses from exiting the dressing at the periphery and better directs diffusing gasses through a substantially centered cutout or window 108. Barrier layer 107 is substantially thin and pliable. A side view of layer 107 is illustrated at right. Cutout 108 enables diffusing gasses to pass through to the adhesive layer and hydrocolloid layers previously outlined.

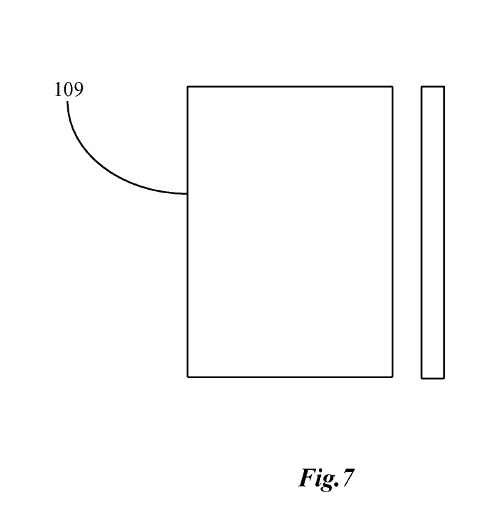

FIG. 7 is a plan view of an oxygen release layer 109 of the hydrocolloid dressing of FIG. 1. Oxygen release layer 109 is a blown fibrous material, sometimes referred to as “Buff Puff” in the medical industry. Oxygen release layer 109 forms the heart of hydrocolloid dressing 100 in terms of its diffusion capabilities. Layer 109 may be saturated with oxygen gas, for example, and may release the oxygen. Peeling off the liner covering to expose the dermal layer of the dressing activates the dressing. In one embodiment, the dressing may be activated once it is removed from a barrier pouch that maintains the saturation level of the dressing under a small amount of pressure.

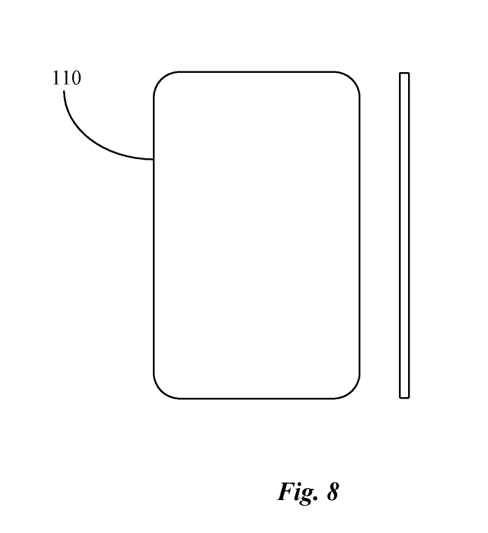

FIG. 8 is a plan view of an absorbent layer 110 of the hydrocolloid dressing of FIG. 1. Absorbent layer 110 may comprise any medical grade materials that function to absorb exudate. Absorbent layer 110 functions to direct exudate from the wound and into the dressing without interrupting the diffusion process. Layer 110 is illustrated in side view to show substantial thickness of the material. In this example, material 110 has corners that are rounded to a 0.25 inch radius.

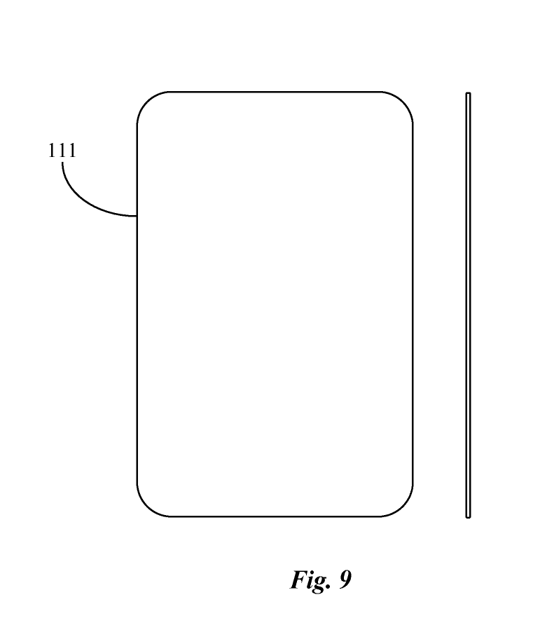

FIG. 9 is a plan view of a second barrier layer 111. Barrier layer 111 is a non-permeable layer representing one wall of the diffusion reservoir containing the oxygen release layer and absorbent layer. Barrier layer 111 may be a poly-based film that is thin durable and pliable.

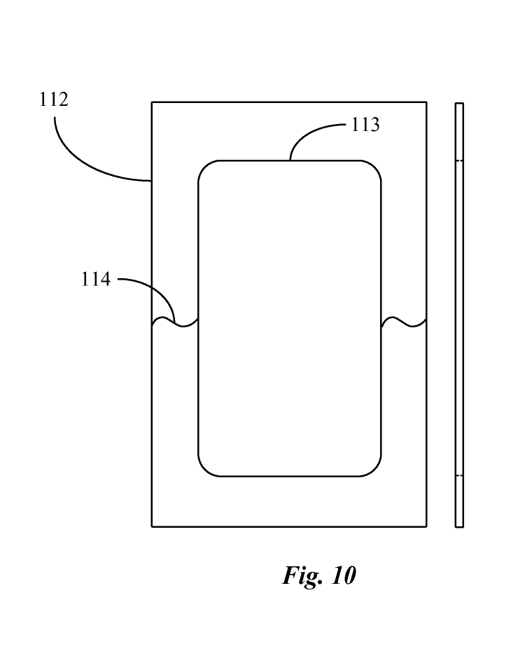

FIG. 10 is a plan view of a second adhesive layer of the hydrocolloid dressing of FIG. 1. Adhesive layer 112 has an adhesive side facing the hydrocolloid layer of the oxygen diffusion dressing. A cutout 113 provided about center through the adhesive layer may provide a transparent view into the absorbent layer to determine if sufficient absorption of exudate is occurring.

Second barrier layer 111 provides a non-permeable barrier to back leaking of therapeutic gases. The adhesive side of layer 112 faces and adheres to the second barrier layer. Both the second barrier layer and the second adhesive layer may be non-permeable layers. In one embodiment, one or more of the layers used to create hydrocolloid diffusion dressing 100 is pre-treated with a therapeutic agent like silver oxide, for example.

It will be apparent to one with skill in the art that the hydrocolloid diffusion dressing of the invention may be provided using some or all of the mentioned features and components without departing from the spirit and scope of the present invention. It will also be apparent to the skilled artisan that the embodiments described above are specific examples of a single broader invention that may have greater scope than any of the singular descriptions taught. There may be many alterations made in the descriptions without departing from the spirit and scope of the present invention.

What is claimed is:

1. A diffusion dressing comprising:
a paper liner;
a hydrocolloid layer;
a first adhesive layer;
a first barrier layer with a center cutout;
an oxygen release layer;
an absorbent layer;
a second barrier layer; and
a second adhesive layer with a center cutout.

2. The diffusion dressing of claim 1, used for diffusing oxygen into a wound.

3. The diffusion dressing of claim 1, wherein the paper liner is removed to expose the hydrocolloid layer before application.

4. The diffusion dressing of claim 1, used as a cover dressing for gauze, alginate, or hydrogel dressings.

5. The diffusion dressing of claim 1, wherein one or more of the layers is perforated to regulate diffusion rate.

6. The diffusion dressing of claim 1, wherein one or more of the layers is pre-treated with silver oxide.

7. The diffusion dressing of claim 1, wherein the paper liner and the hydrocolloid layer are perforated, the perforations symmetrically aligned between the layers.

8. The diffusion dressing of claim 7, wherein the perforations in the hydrocolloid layer are larger in diameter than the perorations in the paper liner.

9. The diffusion dressing of claim 1, vacuum evacuated and oxygen flushed within an oxygen barrier pouch before application.

10. The diffusion dressing of claim 1, wherein the first and second barrier layers form an oxygen reservoir.

11. A method for charging an oxygen hydrocolloid diffusion dressing comprising steps:
(a) placing the dressing inside a oxygen barrier pouch;
(b) vacuum evacuating the oxygen barrier pouch with the dressing inside;
(c) flushing the pouch with oxygen; and
(d) translating oxygen from the pouch into the reservoir of the dressing.

12. The method of claim 11, wherein the oxygen hydrocolloid dressing comprises a paper liner; a hydrocolloid layer; a first adhesive layer; a first barrier layer with a center cutout; an oxygen release layer; an absorbent layer; a second barrier layer; and a second adhesive layer with a center cutout.

13. The method of claim 11, wherein in step (b), a vacuum sealing machine is used to evacuate and flush the pouch with oxygen.

14. The method of claim 12, wherein one or more of the layers is perforated to regulate diffusion rate.

15. The method of claim 12, wherein one or more of the layers is pre-treated with silver oxide.

16. The method of claim 12, wherein the paper liner and the hydrocolloid layer are perforated, the perforations symmetrically aligned between the layers.

17. The method of claim 12, wherein the perforations in the hydrocolloid layer are larger in diameter than the perorations in the paper liner.

18. The method of claim 12, wherein in step (d), gas from the pouch enters the reservoir of the dressing through one or more gas passages.
